(19) United States
(12) Patent Application Publication
(10) Pub. No.: US 2002/0151770 A1
(43) Pub. Date: Oct. 17, 2002

(54) Implantable medical device with sensor

(76) Inventors: Austin F. Noll III, Santa Barbara, CA (US); Keith Alan Miesel, St. Paul, MN (US); Lee Stylos, Stillwater, MN (US); Mark Allen Geiger, Ventura, CA (US); Curtis D. Kinghorn, Lino Lakes, MN (US); Michael Eugene Leckrone, Mahtomedi, MN (US)

Correspondence Address: Curtis D. Kinghorn, Medtronic, Inc., 710 Medtronic Parkway N.E., Minneapolis, MN, 55432, US

(21) Appl. No.: 10/041,431

(22) Filed: Jan. 4, 2002

Related U.S. Application Data
(60) Provisional application No. 60/259,746, filed on Jan. 4, 2001.

(51) Int. Cl. A61B 5/00

(52) U.S. Cl. 600/300; 128/903

(57) ABSTRACT

A device and method for measuring and communicating parameters of a brain, tissue or other organs is disclosed. The invention includes a sensor to sense the parameter of interest and then communicate the sensed parameter to an activation system. The activation system may cause the parameter to be displayed, processed or cause action to be taken. The activation system may be entirely or partially implanted or entirely external to the patient.

### FIELD OF THE INVENTION

The present invention relates to a device for measuring and communicating parameters of a brain, tissue or other organs.

### BRIEF DESCRIPTION OF RELATED ART

It is often desirable to measure and communicate parameters of a brain, tissue or other organs. For example, many people have a condition called hydrocephalus. Hydrocephalus is a condition of excessive accumulation of CSF in the ventricles or brain cavities. Hydrocephalus can result from congenital conditions interfering with normal CSF circulation or as the result of a problem with CSF re-absorption. A typical adult has a total of about 120-150 cc of (cerebrospinal fluid) CSF with about 25 cc in the ventricles in the brain. A typical adult also produces about 500 cc/day of CSF, all of which is reabsorbed into the blood stream on a continuous basis.

Different conditions can cause the CSF pressure to vary, often in an increasing and dangerous manner. Excessive accumulation of CSF due to hydrocephalus causes increased pressure upon the brain. Whatever the cause, over time, this increased CSF pressure causes damage to the brain tissue. It has been found that shunting the excess CSF to another area of the body is therapeutically beneficial and generally allows the patient to lead a full and active life. Both on a short and long term basis, many physicians believe that it is desirable to measure and read the CSF fluid pressure.

Further, many physicians believe it is desirable to monitor both the physiologic parameters associated with various medical conditions but also the reaction of various organs and tissue to treatments administered or directed by the physicians. Unfortunately, there has not been many devices or methods available to physicians to monitor physiologic parameters, particularly on a continuing basis. What systems that are available often require wires or tubes running from sensors within the patient's body to the outside world. These wires and tubes increase the likelihood of infection and irritation. Therefore, there is a need for a system whereby implantable sensors may communicate with the outside world without requiring wires, tubes or the like to pass from within the patient's body to outside the patient's body.

### SUMMARY OF THE INVENTION

A device and method for measuring and communicating parameters of a brain, tissue or other organs is disclosed. The invention includes a sensor to sense the parameter of interest and then communicate the sensed parameter to an activation system where the parameter may be displayed, processed or cause action to be taken. The activation system may include an external device. The present invention allows chronic and stable measurement and communication of parameters, including parameters, to be made.

In an embodiment, the device measures and communicates parameters of a brain, tissue or other organs. The invention includes a sensor. The sensor in one embodiment is located at the distal end of a probe and is preferably placed in the area of the brain, tissue or other organ where a measurement is desired. In another embodiment, the sensor is co-located with an implanted device in the tissue or organ of interest. In a further embodiment, the sensor is located in or near the tissue or organ of interest.

In one embodiment, the sensor is part of a passive system that allows parameter measurements to be made and communicated to an attending practitioner when the passive system receives power from an external source. The part of the passive system that receives power from the external source and communicates pressure measurements is preferably located on or next to the skin of the patient while the sensor is located near or at the area where a measurement is desired to be made.

The passive system couples to an external device that provides power to the passive system. This power is used to power the sensing operation of the sensor and to upload the sensed information from the passive system to an external device, if desired. As a result, when coupled to the external power source, the passive system is able to measure and uplink measured parameters from the sensor to an external device or to power the operation of an implanted device.

In an alternate embodiment, the sensor is part of a system having a long-term energy source and storage system that allows parameter measurements to be taken periodically or upon demand, stored and then communicated to an attending practitioner as desired. The part of the system that provides power, stores parameter measurements and communicates the parameter measurements is preferably located on or next to the skin of the patient or may be embedded in the bone of the patient such as in the skull or pelvis.

The long-term energy source may be rechargeable. This power from the long-term energy source is used to power the sensing operation of the sensor, store the parameter measurements and to upload the sensed parameter information from the system to an external device or to an implanted device.

In an alternate embodiment of the invention, the sensed parameter is used to control a medical device such as a pump or valve in a CSF drainage system, a drug delivery system or an electrical stimulation device. These medical devices may be wholly or partially implanted.

It is an object of one embodiment of the invention to provide a system for measuring a parameter that does not require a continuous source of power such as a battery or power capacitor.

It is another object of one embodiment of the invention to provide a device that communicates a sensed parameter to an external device.

In another embodiment of the invention, it is an object of the invention to provide a system that stores sensed parameter measurements to be uploaded to an external device at a later time.

In a further alternate embodiment of the invention, it is an object of the invention to provide a system that actively responds to a sensed parameter to take an action.

In another alternate embodiment of the invention, it is an object of the invention to provide a sensor of parameters that requires the tissue of interest to be exposed only once during implantation for implantation of the sensor and is thereafter not exposed while providing long-term monitoring of the parameter of interest.

It is an object of the invention in another embodiment of the invention to provide an implantable device that provides information about a parameter of interest which does not depend on a battery to operate and therefore does not depend on the battery life to remain in operation.

These and other objects of the invention will be clear from the description of the invention contained herein and more particularly with reference to the Figures. Throughout the description, like elements are referred to by like reference numbers. Further, it is clear that to changes to the description contained herein may occur to those skilled in the art and still fall within the scope of the invention.

### BRIEF DESCRIPTION OF THE DRAWINGS

FIG. 1 is a block diagram of the invention.

FIG. 2 is a block diagram of one embodiment of the invention.

FIG. 3 is a side view of one embodiment of the invention.

FIG. 4 is a side cross-sectional view of the embodiment of FIG. 3.

FIG. 5 is a side cross-sectional view of the embodiment of FIG. 3 in place in a skull.

FIG. 6 is a side cross-sectional view of an alternate embodiment of the invention in place in a skull.

FIG. 7 is a side cross-sectional view of an alternate embodiment of the invention in place on a skull.

FIG. 8 is a perspective view of an alternate embodiment of the invention.

FIG. 9 is a perspective view of another alternate embodiment of the invention.

FIG. 10 is a side cross-sectional view of another alternate embodiment of the invention.

FIG. 11 is a schematic drawing of one embodiment of the invention.

FIG. 12 is a schematic drawing of another embodiment of the invention.

FIG. 13 is a schematic drawing of another embodiment of the invention.

FIG. 14 is a chart showing the charging and transmitting sequence of one embodiment of the invention.

FIG. 15 is a schematic drawing of another embodiment of the invention.

FIG. 16 is a block diagram of an alternate embodiment of the invention.

FIG. 17 is a schematic drawing of another embodiment of the invention.

FIG. 18 is a schematic drawing of another embodiment of the invention.

### DETAILED DESCRIPTION OF THE INVENTION

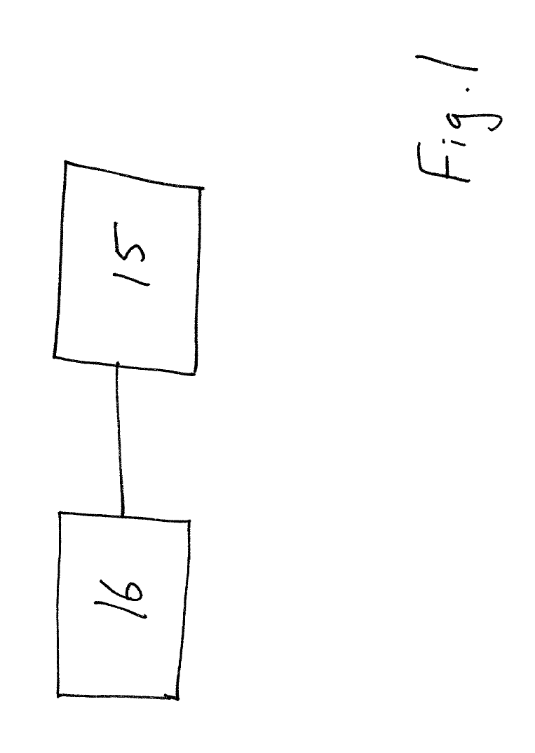

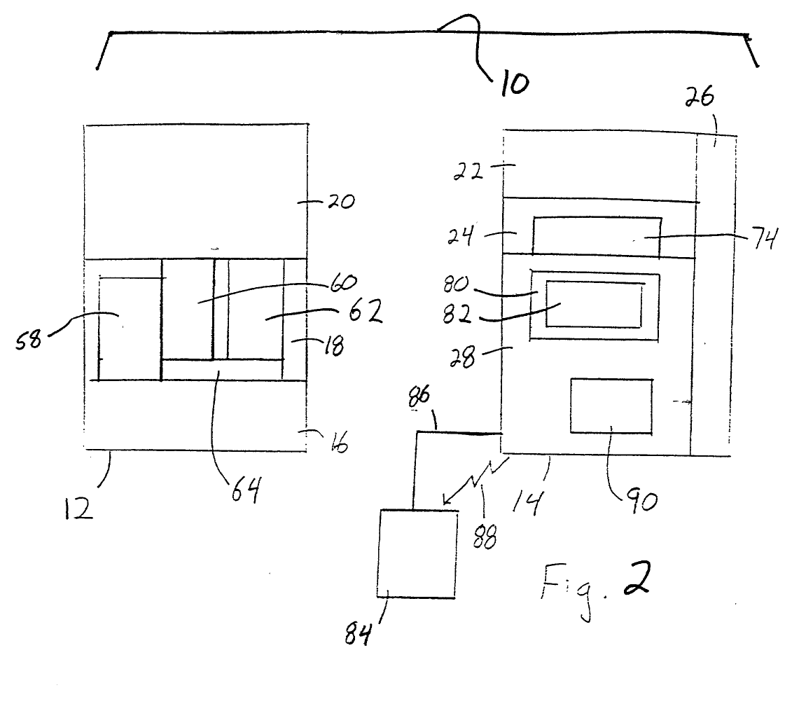

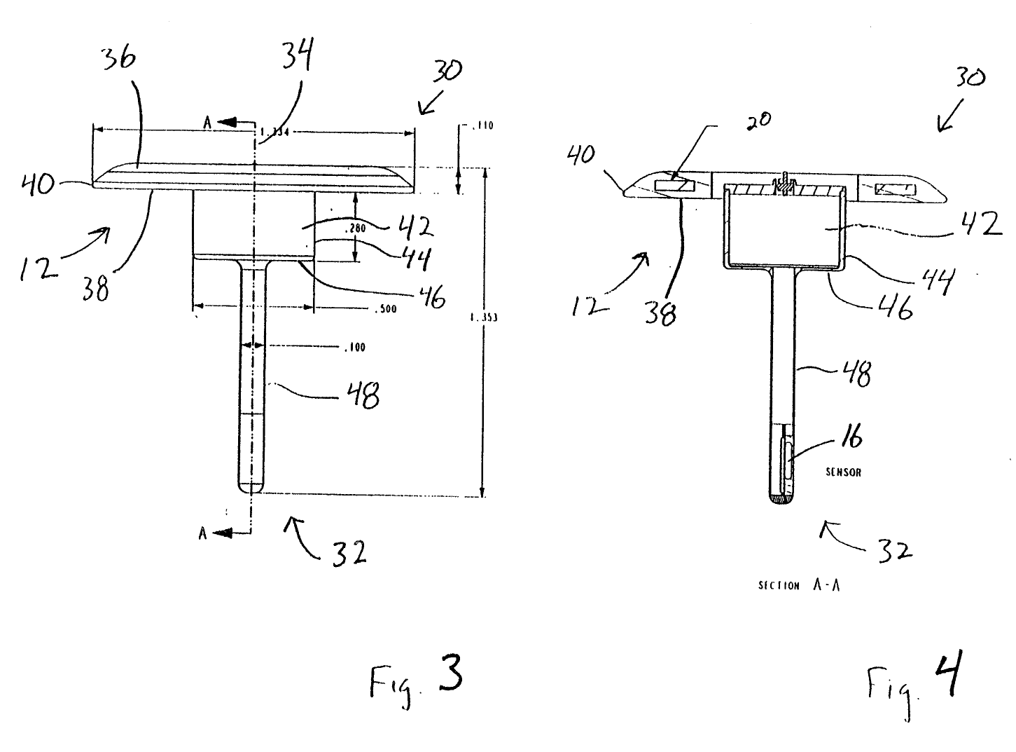

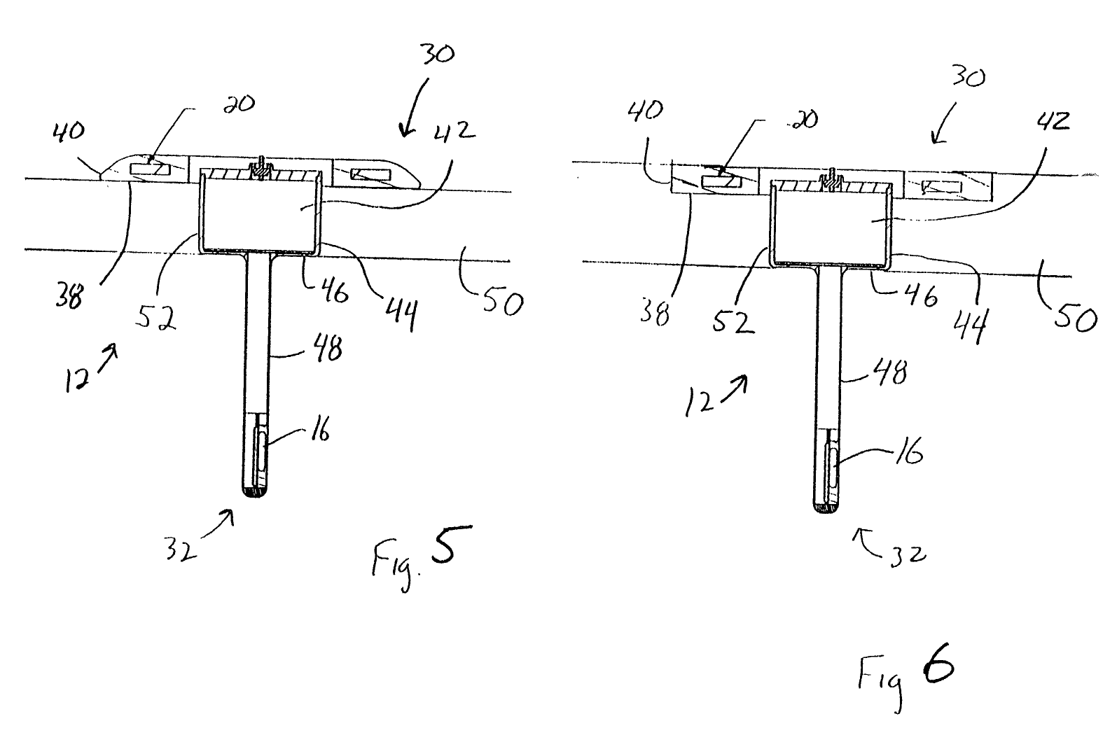

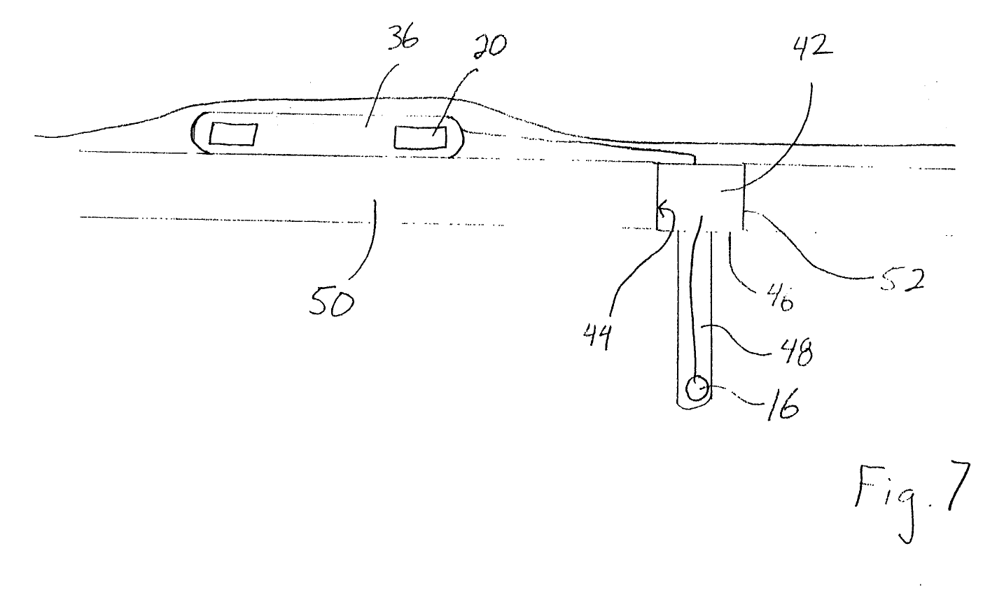

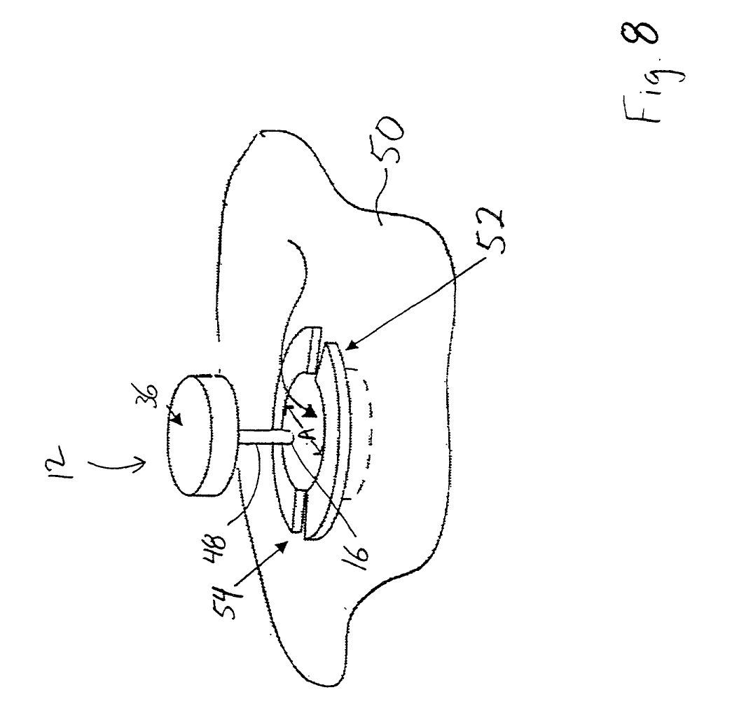

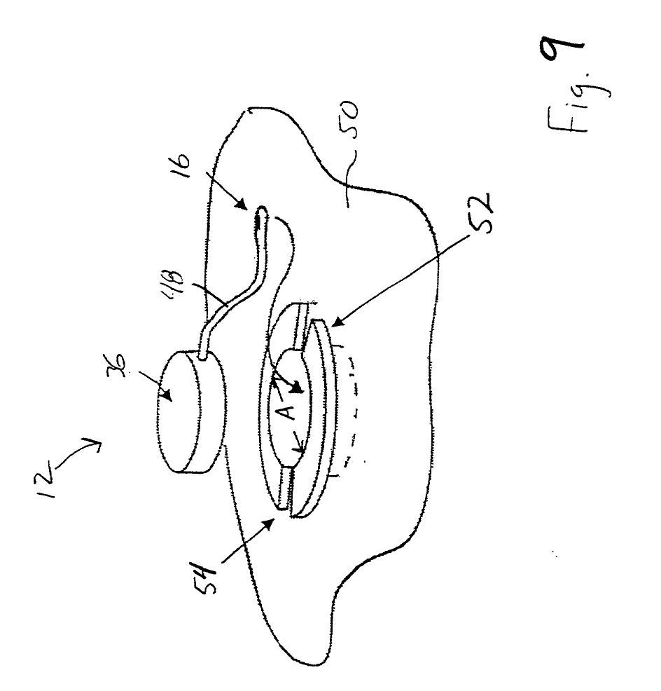

The device embodying the present invention is shown in FIG. 1 generally labeled 10. The device 10 includes a sensor 16 and an activation system 15 connected to and responding to sensor 16. Sensor 16 is implanted in the body of a patient. In one embodiment, activation system 15 includes an external device 14 that is located outside the body of the patient. In another embodiment, activation system 15 and sensor 16 are co-located in the body of the patient. In a further embodiment, activation system 15 and sensor 16 are both implanted in a patient but are not co-located.

Activation system 15 preferably includes a user communication system 28. User communication system 28 may be entirely implanted within the patient of may have a portion that is located in external device 24. In the embodiment where activation system 15 includes an external device 14, external device 14 may include a portion of the user communication system 28 that includes an external coil 22, external electronics 24 and a power source 26.

User communication system 28 is in communication with sensor 16 and may communicate information from or about sensor 16 or may communicate alarms or other information based on the values that sensor 16 detects. Further, user communication system 28 may communicate information that is processed from information provided by sensor 16 such as trends in the sensed parameters from sensor 16.

In one embodiment, user communication system 28 includes a receiver 74. User communication system 28 preferably includes a display system 80 that displays or otherwise communicates the parameter information received from a transmitter 60 by receiver 74 to a user. User communication system 28 may include a display screen 82 that displays the parameter information to the physician or other user. Alternately, user communication system 28 may pass the parameter information from the external device 14 to an external computer 84, the internet or through a modem by direct connection 86 or through telemetry 88 as is well understood in the art. Computer 84 can display the parameter information on its display screen 82, record the information or further process the information. If the information is passed through the internet or through a modem, the information may be remotely used, processed or displayed as desired.

User communication system 28 may also include an alarm 90 that is part of the external device 14 or the external computer 84 that is triggered to alert the user to a parameter that is outside of a pre-determined range. The alarm 90 can also take the form of an audible or visible warning such as a warning chime or a flashing visual display panel, a physical warning such as a vibrating alarm or other means of alerting the user or emphasizing the status as will occur to those skilled in the art.

Activation system 15 may also direct components to take action based on the information from or about sensor 16 or processed from information provided by sensor 16. For example, activation system 15 may direct a drug pump to provide a certain amount of a drug to the patient or may direct an electrical stimulation device to stimulate the patient.

All or part of activation system 15 may be implanted in the patient or may be entirely located outside the patient. Where activation system 15 includes at least a part outside the patient, the outside part of activation system 15 is an external device 14.

Sensor 16 may be of the type that can detect any or all of the following, as is well understood in the art. Examples of such sensors 16 are given below. However, it is to be understood that the descriptions below are not intended to limit the type of sensor 16 or the specific structure of sensor 16. It is clear that sensors 16 of the types listed below are well understood to those skilled in the art and the specific types and uses of sensors 16 will be immediately clear to those skilled in the art. It is also clear that the form or structure of the sensor 16 is not critical to the instant invention so long as such sensor 16 can be implanted and used as is described herein.

| Sensor | |
|---|---|
| Type | Examples: |
| Respir- | (1) Piezoelectric activity sensors: U.S. Pat. No. 4,485,813 |
| ation | issued to Anderson et al.; 4,576,179, 3,782,368, |
| | 4,169,462, 4,185,621 and 4,443,730. These typically |
| | describe the use of piezo transducers that are sensitive to |
| | body breathing motions as well as impacts or forces |
| | incident from various directions. |
| | (2) U.S. Pat. No. 5,195,529, issued Mar. 23, 1993 to Lauri |
| | Malkamaki entitled “SENSOR FOR MONITORING |
| | RESPIRATION |
| | (3) U.S. Pat. No. 6,064,910 issued May 16, 2000 entitled |
| | “RESPIRATOR RATE/RESPIRATION DEPTH |
| | DETECTOR AND DEVICE FOR MONITORING |
| | RESPIRATORY ACTIVITY EMPLOYING SAME” to Jonas |
| | Andersson, Johan Lidman and Carolina Bigert |
| Electrolyte | U.S. Pat. No. 5,234,567 entitled “Gas Sensor” issued |
| concen- | Aug. 10, 1993 to Bryan S. Hobbs and Yat S. Chan and |
| tration | U.S. Pat. No. 5,746,898 issued May 5, 1998 to Walter |
| | Preidel. |
| O₂ | U.S. Pat. No. 6,267,724 entitled “Implantable Diagnostic |
| | Sensor” issued on July 31, 2001 to David W. Taylor and |
| | U.S. Pat. No. 6,201,980 entitled “Implantable Medical |
| | Sensor System” issued on March 13, 2001 to Christopher |
| | B. Darrow, Joe H Satcher, Jr., Stephen M. Lane, |
| | Abraham P. Lee and Amy W. Wang. |
| PH | As is understood in the art |
| glucose | U.S. Pat. No. 4,703,756 issued Nov. 3, 1987 entitled |
| | “Complete Glucose Monitoring System with an |
| | Implantable, Telemetered Sensor Module” to David A |
| | Gough, Joseph Y. Lucisano, Jon C. Armour and Brian |
| | DMcKean; |
| Blood | As is understood in the art |
| pressure | |
| Intra- | (1) U.S. Pat. No. 4,984,567 issued Jan. 15, 1991 entitled |
| cranial | “APPARATUS FOR MEASURING INTRACRANIAL |
| Pressure | PRESSURE” to Naoki Kageyama, Hiroji Kuchiwaki, Junki |
| | Ito, Nobumitsu Sakuma, Yukio Ogura; U.S. Pat. No. |
| | 4,971,061 issued Nov. 20, 1990 entitled “APPARATUS |
| | FOR RECORDING INTRACRANIAL PRESSURE” to |
| | Naoki Kageyama, Hiroji Kuchiwaki, Nissincho Aichi-gun, |
| | Junki Ito, Nobumitsu Sakuma, Yukio Ogura, Eiji |
| | Minamiyama, |
| Heart rate | (1) ECG MONITORS--require at least two electrodes to |
| | be attached to the body, as described in U.S. Pat. Nos. |
| | 4,630,614 and 4,269,195. |
| | (2) ULTRASOUND--Must be attached to the chest. |
| | (3) ACOUSTIC--Must be attached to the chest, or nostril |
| | as in U.S. Pat. Nos. 4,421,113 4,281,651. |
| | (4) INFRARED--Which is attached to the finger. Nothing is |
| | known of the remote (detached) passive heart rate |
| | monitors. |
| | (5) A sensor that detects air flow through the nostril. As in |
| | U.S. Pat. No. 4,036,217. |
| | (6) A strap that is stretched across the chest to detect the |
| | chest movements during respiration. As in U.S. Pat. Nos. |
| | 3,802,419 and 4,540,002. |
| | (7) A device attached to the mattress U.S. Pat. No. |
| | 3,325,799. As in U.S. Pat. Nos. 4,146,885; 4,066,072; |
| | 3,631,438; and France PV No. 43858 No. 1,480,160; |
| | (8) A CO2 sensor that is attached to the breathing system. |
| | (9) A Thermistor sensor that is attached to the breathing |
| | system. |
| | (10) Ultrasound or microwave Doppler radar (invasive due |
| | to radiation). As in U.S. Pat. Nos. 3,875,929; 3,796,208. |
| | (11) Inductance change respiratory electrodes. As in U.S. |
| | Pat. Nos. 3,911,899; 3,658,052. |
| temper- | (a) U.S. Pat. No.: 5,596,995 issued Jan. 28, 1997 |
| ature | entitled “BIOMEDICAL DEVICE HAVING A |
| | TEMPERATURE SENSING SYSTEM” to Marshall L. |
| | Sherman, Thomas M. Castellano, Jose J. Moya, |
| | (b) thermocouple |
| activity/ | U.S. Pat. No. 5,593,431 entitled “Medical service |
| posture | employing multiple DC accelerometers for patient activity |
| | and posture sensing and method” issued Jan. 14, |
| | 1997 to Todd J. Sheldon and U.S. Pat. No. 5,031,618 |
| | entitled “Position-responsive neuro stimulator” issued July |
| | 16, 1991 to Keith R. Mullett. |
| eeg | (a) January 1973 edition of Popular Electronics magazine |
| | at pages 40-45. |
| | (b) U.S. Pat. No.: 5,215,086 issued Jun. 1, 1993 |
| | entitled “THERAPEUTIC TREATMENT OF MIGRAINE |
| | SYMPTOMS BY STIMULATION” by Reese S. Terry, Jr., |
| | Joachim F. Wernicke, Ross G. Baker, Jr., |
| | (c) U.S. Pat. No. 4,744,029 issued May 10, 1988 |
| | entitled “BRAIN ELECTRICAL ACTIVITY ANALYSIS |
| | AND MAPPING” to Gabriel Raviv, Gil Raviv, |
| | (d) U.S. Pat. No.: 4,753,246 issued Jun. 28, 1988 |
| | entitled “EEG SPATIAL FILTER AND METHOD” by |
| | Walter J. Freeman |
| | (e) U.S. Pat. No. 4,974,602 issued Dec. 4, 1990 entitled |
| | “ARRANGEMENT FOR ANALYZING LOCAL |
| | BIOELECTRIC CURRENTS IN BIOLOGICAL TISSUE |
| | COMPLEXES” to Klaus Abraham-Fuchs Gerhard |
| | Roehrlein, Siegfried Schneider, |
| ecg | As is understood in the art |
| emg | (a) U.S. Pat. No.: 4,753,246 issued Jun. 28, 1988 |
| | entitled “EEG SPATIAL FILTER AND METHOD” to Walter |
| | J. Freeman |

In use, sensor 16 is preferably placed in or in contact with the tissue, organ or other part of the body where parameter information may be sensed. For example, sensor 16 may be placed in or in contact with the spinal column, organs of the body such as the liver, kidneys, the heart, the bladder, tumors or growths, body tissue, joints, cavities, sinuses or spaces between organs or tissue or other areas as will occur to those skilled in the art.

Sensor 16 may be part of a probe 12 that includes sensor 16, probe electronics 18 and a probe coil 20.

In one embodiment, device 10 has a single sensor 16. However, device 10 may also have multiple sensors 16 sensing the same or different parameters at a single or at multiple locations. Where sensor 16 senses parameters other than physiological, probe may include sensors that sense non-parameters in addition to or in any combination with the sensors to sense parameters.

In the embodiment of the invention shown in FIGS. 3 and 4, probe 12 has a proximal end 30 and a distal end 32 and a central axis 34. Sensor 16 is preferably located at the distal end 32.

A probe head 36 is located at the proximal end 30. In one embodiment, probe head 36 is roughly discoid in shape and includes an embedded probe coil 20. Probe coil 20 is an inductive coil. In the embodiment shown in FIGS. 3 and 4, probe coil 20 is wound around axis 34 in the plane of probe head 36. Probe head 36 includes an underside 38 and an outer edge 40.

In the embodiment of FIGS. 3 and 4, the probe electronics 18 are stored in an electronics case 42, attached to the underside 38 of probe head 36. The electronics case 42 has a periphery 44 and an underside 46. Electronics case 42 is preferably cylindrical with a smaller diameter around axis 34 than has probe head 36.

As shown in FIGS. 3 and 4, sensor 16 is separated from electronics case 42. This is preferably accomplished by locating sensor 16 at the distal end of a body 48 connected to the underside 46 of electronics case 42. Body 48 may be made of a stiff material such as titanium or a rigid body-compatible plastic like polyurethane. Alternately, body 48 may be made of a flexible material such as a flexible body-compatible plastic such as polyurethane that is inherently flexible by its composition or designed to be flexible by its structural design. In either the rigid or flexible case, the material of body 48 may be any metal, plastic, ceramic or other material that is body-compatible and is as flexible or rigid, in varying degrees, as desired as is well understood in the art.

In the embodiment where body 48 is rigid, sensor 16 will be located at a fixed location relative to the electronics case 42. Where body 48 is flexible, such as in the embodiment shown in FIG. 9, sensor 16 may be placed where desired in the brain, tissue or in other organs wherever in the body. In particular, where body 48 is flexible, sensor 16 may be placed where the distance from sensor 16 to the electronics case 42 varies as, for example, with movement.

In addition, where body 48 is flexible, sensor 16 may be placed on or in areas where it would be difficult to place sensor 16 where body 48 to be rigid. For example, where body 48 is flexible, sensor 16 may be “slid” between the dura and the skull to a desired position between the dura and the skull. Other locations to locate the sensor will occur to those skilled in the art.

In a further alternate embodiment, sensor 16 may be connected to electronics case 42 through a system known as a “body bus”. The “body bus” is a telemetry system where the patient's own body provides the interconnection between the sensor 16 and the electronics case 42. An example of such a “body-bus” communication system is given in U.S. Pat. Nos. 4,987,897 and 5,113,859, issued to Hermann D. Funke on Jan. 29, 1991 and May 19, 1992, entitled “Body Bus Medical Device Communication System” and “Acoustic Body Bus Medical Device Communication System” respectively, the teachings of which are incorporated herein by reference in its entirety. Alternately, a radio frequency telemetry approach as described in U.S. Pat. No. 5,683,432 to Goedeke may be used to link sensor 16 to electronics case 42.

The sensor 16 is preferably calibrated at the manufacturing site by comparing its measurements with measurements from a standardized sensor. Calibration coefficients, which are unique to each sensor 16, are computed and stored in the external device 14, probe electronics 18, storage device 78 or microprocessor 102 for the purpose of post-measurement processing to achieve an accurate report of the parameters measured by sensor 16.

Because probe 12 will be inserted into the body, probe 12 should be hermetically sealed to prevent the intrusion of body fluids into probe 12.

In the embodiment shown in FIGS. 3 and 4, the proximal end 30 is located either immediately outside of or incorporated into the skull 50 of the patient or in other bony places such as the pelvis, sternum, scapula, to name but a few places that will occur to those skilled in the art. This is preferably accomplished by making the probe head 36 with a larger diameter around axis 34 than the electronics case 42 has. Then, for example, to place the probe 12 in the skull 50, a hole 52 is drilled in skull 50 having a diameter about the same as the diameter of electronics case 42 (FIG. 5). Hole 52 should go entirely through the skull 50 and have the same diameter as the diameter of electronics case 42. The sensor 16 and body 48 of probe 12 is placed through the hole 52 until the electronics case 42 contacts hole 52. Electronics case 42 is then aligned with hole 52 and pushed through hole 52 until the underside 38 of probe head 36 contacts the skull 50. Electronics case 42 should be dimensioned so as not to extend entirely through hole 52.

Alternately, screw threads may be placed around the periphery 44 of electronics case 42. In this embodiment, hole 52 is a threaded hole with threads matching the threads of electronics case 42. Electronics case 42 is brought into contact with hole 52 as described above. However, instead of pushing electronics case 42 through hole 52, electronics case 42 is threaded into hole 52.

In a further alternate embodiment shown in FIG. 6, screw threads are placed on the outer edge 40 of probe head 36. Hole 52 is dimensioned to have a diameter approximately the same as the diameter of probe head 36. In this embodiment as well, hole 52 has threads corresponding to the threads on probe head 36. To place the probe 12, the sensor 16, body 48 and electronics case 42 of probe 12 is placed through the hole 52 until the outer edge 40 of probe head 36 contacts hole 52. Probe head 36 is then aligned with hole 52 and threaded through hole 52 until probe head 36 has a desired orientation, such as flush with the skull 50. In this embodiment, electronics case 42 may or may not have the same diameter as probe head 36.

In another embodiment shown in FIG. 7, probe head 36 is separated from, although connected to, electronics case 42. In this embodiment, electronics case 42 is mounted through a hole 52 bored in, for example, the skull 50, and body 48 with sensor 16 is still attached to electronics case 42 in all the variants described herein. But, in this embodiment, probe head 36 with probe coil 20 is implanted underneath the patient's skin but above or in the skull 50. Probe head 36 may be attached to the patient's skull 50 by screws, adhesives or other means that will occur to those skilled in the art. Alternately, a separate hole from hole 52 may be bored into the skull 50 to receive the probe head 36. Where probe head 36 is located in the separate hole, probe head 36 may have screw threads placed on the outer edge 40 of probe head 36 and the separate hole is dimensioned to have a diameter approximately the same as the diameter of probe head 36 with threads corresponding to the threads on probe head 36. In this embodiment, to place the probe 12, the sensor 16, body 48 and electronics case 42 of probe 12 is placed through the hole 52. Probe head 36 is then attached to the skull 50 as described above.

In a further embodiment shown in FIGS. 8 and 9, a burr-hole ring 54 having an opening 56 with a diameter “A” is placed in a hole 52 in skull 50. Burr-hole ring 54 may be screwed into the bone of the skull 50 or otherwise attached to the skull 50 in a fashion well known for burr-hole rings. In this embodiment, probe head 36 has a diameter about equal to the diameter “A” of the opening 56 of burr-hole ring 54. Probe head 36 is placed in the opening 56 where it may be held in place by means such as friction, body-compatible adhesive or other means that will occur to those skilled in the art.

In the embodiment of FIG. 8, the body 48 is rigid so that sensor 16 is located a fixed distance from and at a fixed relationship to the probe head 36. In the embodiment of FIG. 9, body 48 is flexible. In this embodiment, the sensor 16 is placed through the opening 56 to a desired location.

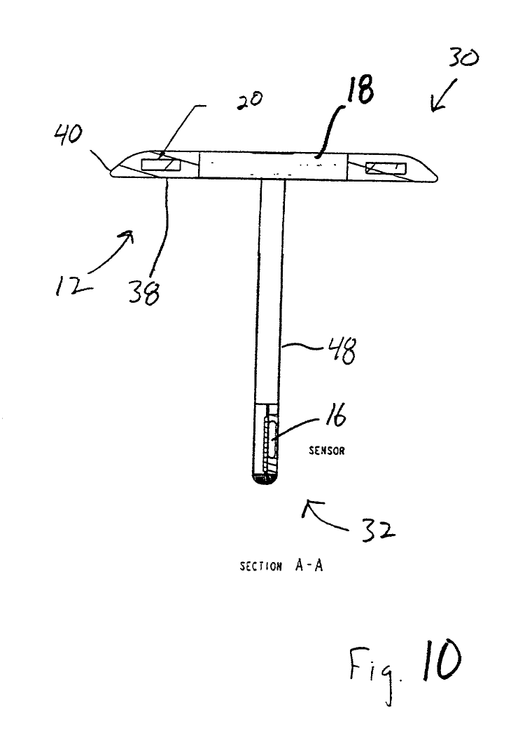

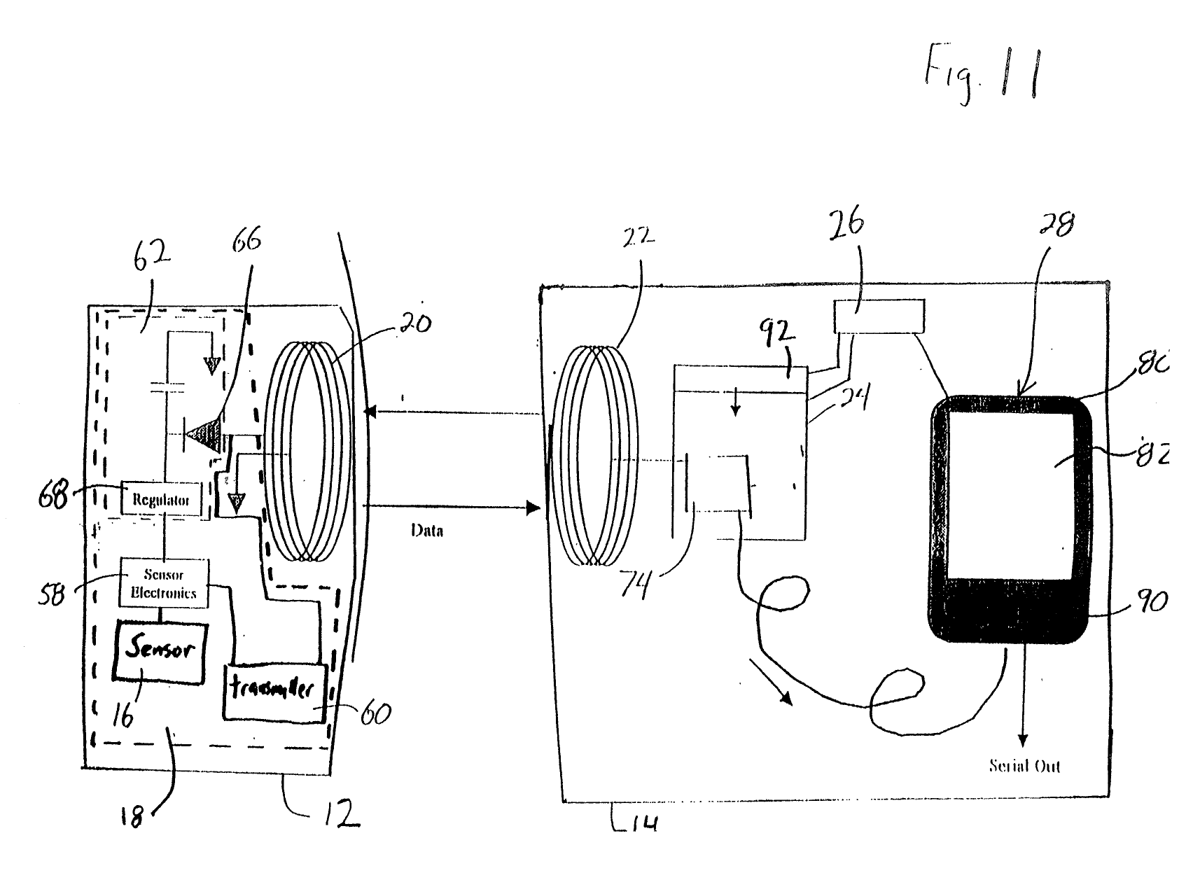

In a further embodiment shown in FIG. 10, the probe head 36 may contain all or part of the probe electronics 18. In this embodiment, there may be no need to have an electronics case 42. Therefore, the sensor 16 may be attached directly to probe head 36 through a rigid or flexible body 48. In use, for example to place the device in the head, a hole 52 is drilled through skull 50 and sensor 16 placed through hole 52 to a desired location. Then, probe head 36 may be attached to the skull 50 as described above.

In a variant of this embodiment, the probe head 36 may be located a distance from the hole 52. For example, the probe head 36 may be located under the skin near the clavicle or in the abdomen at sites common for placing RF powered implantable neurological stimulators. In this embodiment, it may be necessary to use a burr-hole ring 54 to position the body 48 at the skull 50 so that sensor 16 will not move with respect to the hole 52. Further, the probe electronics may also be located in total or in part in the body 48 in any of the embodiments described herein.

Probe electronics 18 includes sensor electronics 58 and a transmitter 60. Sensor electronics 58 is connected to sensor 16 and provides power to sensor 16, directs sensor 16 to take measurements, processes the sensed measurement signal from sensor 16 and converts the sensed signal to a digital signal. This digital signal is preferably passed to transmitter 60.

Transmitter 60 is connected to sensor electronics 58 and probe coil 20. Probe coil 20 acts as an antenna as will be explained hereafter. Transmitter 60 and probe coil 20 communicate parameter information determined by sensor 16 to the external device 14 by telemetry. Examples of telemetry systems are shown in U.S. Pat. No. 5,683,432 entitled “Adaptive, Performance-Optimizing Communication System for Communicating with an Implanted Medical Device”, issued on Nov. 4, 1997 to Steven D. Goedeke, Gregory J. Haubrich, John G. Keimel and David L. Thompson, U.S. Pat. No. 5,752,976 entitled “World Wide Patient Location and Data Telemetry System for Implantable Medical Devices”, issued on May 19, 1998 to Edwin G. Duffin, David L. Thompson, Steven D. Goedeke and Gregory J. Haubrich, U.S. Pat. No. 5,843,139 entitled “Adaptive, Performance-Optimizing Communication System for Communicating with an Implanted Medical Device”, issued on Dec. 1, 1998 to Steven D. Goedeke, Gregory J. Haubrich, John L. Keimel and David. L. Thomson and U.S. Pat. No. 5,904,708 entitled “System and Method for Deriving Relative Physiologic Signals”, issued on May 18, 1999 to Steven D. Goedeke, the teachings of which are incorporated herein in their entireties by reference. Other alternate means of communication between the probe 12 and the external device 14 include amplitude shift keying (ASK), binary phase shift key (BPSK) or quadrature phase shift key (QPSK) to name but a few choices.

In addition, in this embodiment, probe electronics 18 includes an AC/DC conversion system 62 (FIG. 11). Probe coil 20 is connected to AC/DC conversion system 62. Probe electronics 18 allows power to be transferred from the external device 14 to probe 12 to power probe 12 and at the same time allows probe 12 to uplink sensed parameters from probe 12 to external device 14. This simultaneous power transfer and uplink of information is preferably done by a technique known as absorption modulation as is well understood in the art.

In this embodiment, AC/DC conversion system 62 includes a rectifier 66 and a regulator 68. Probe coil 20 will be inductively coupled to an external coil 22 in the external device 14 as will be explained hereafter. This inductive coupling between probe coil 20 and external coil 22 provides power to the probe coil 20. This power will be in the form of an alternating current. In this embodiment, this AC current has a frequency of about 175 kHz although other frequencies may be used as desired.

Rectifier 66 is connected to probe coil 20 and converts the AC power received from the probe coil 20 to DC power. Rectifier 66 is preferably a full-wave rectifier as is well understood in the art but may be other rectification systems as is also well understood in the art. The DC power is passed through the regulator 68 that ensures a relatively constant DC level despite variations in power received from the probe coil 20 due, for example, to the relative movement of the probe coil 20 to the external coil 22. In this way, regulated DC power is provided to power the probe electronics 18.

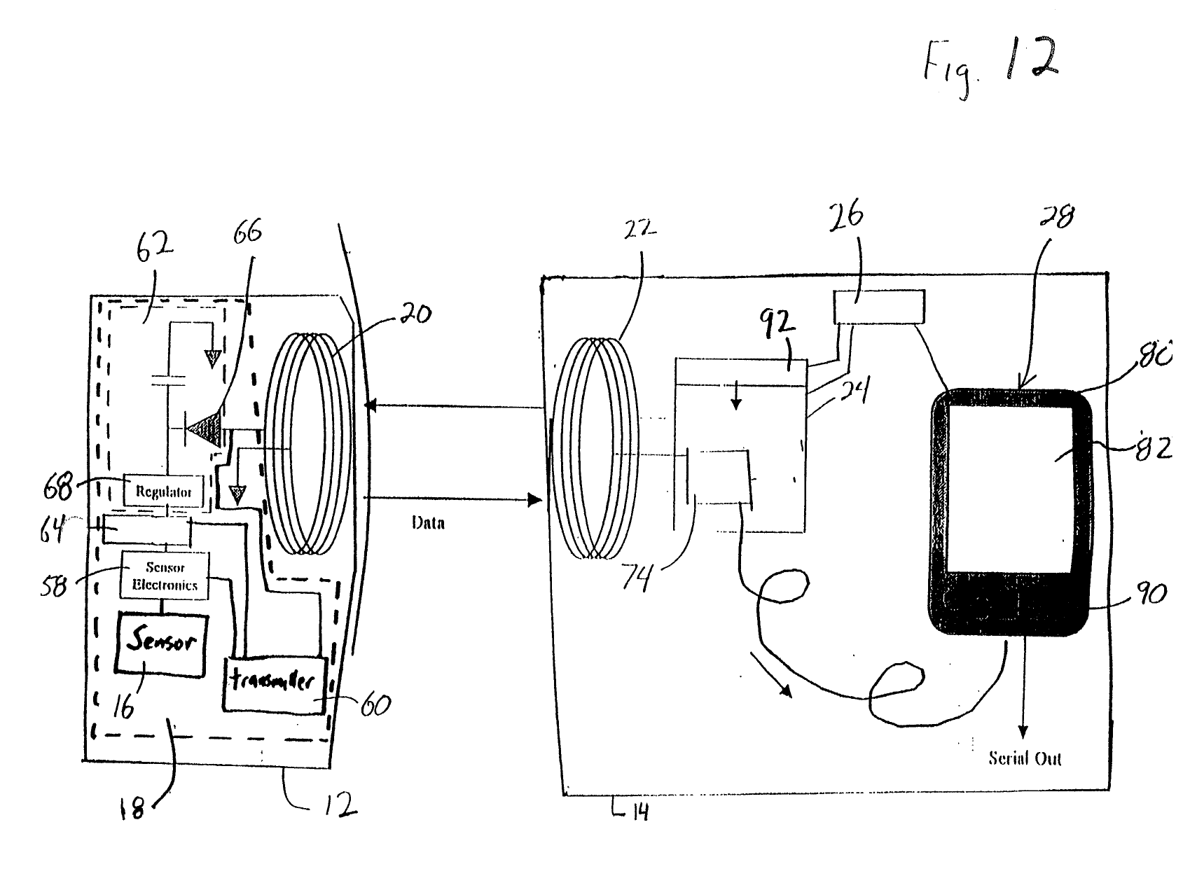

In an alternate embodiment (FIG. 12), probe electronics 18 includes the AC/DC conversion system 62 described above and in addition includes a temporary energy source 64. Probe coil 20 is again connected to AC/DC conversion system 62. Probe coil 20 is inductively coupled to external coil 22.

In this embodiment, a temporary energy source 64 is connected to AC/DC conversion system 62. Temporary energy source 64 preferably takes the form of a rechargeable battery or a power capacitor such as a “super capacitor”, having for example a small capacity such as 1 μf, although larger or smaller capacities may be used as desired and non-rechargeable batteries may also be used.

Inductive coupling between probe coil 20 and external coil 22 provides power to the probe coil 20 and through AC/DC conversion system 62, charges up the temporary energy source 64. Temporary energy source 64 then provides the energy to power the probe electronics 18.

Probe coil 20, in this embodiment (FIG. 11), also acts as an antenna connected to transmitter 60 to transmit information from sensor 16 to the external device 14. In this role, probe coil 20 acts as an antenna in addition to acting as an inductive coil for receiving power from the external device 14 as described above. As described above, probe coil 20 is a coil so that when probe coil 20 acts as an antenna, probe coil 20 is a coil antenna.

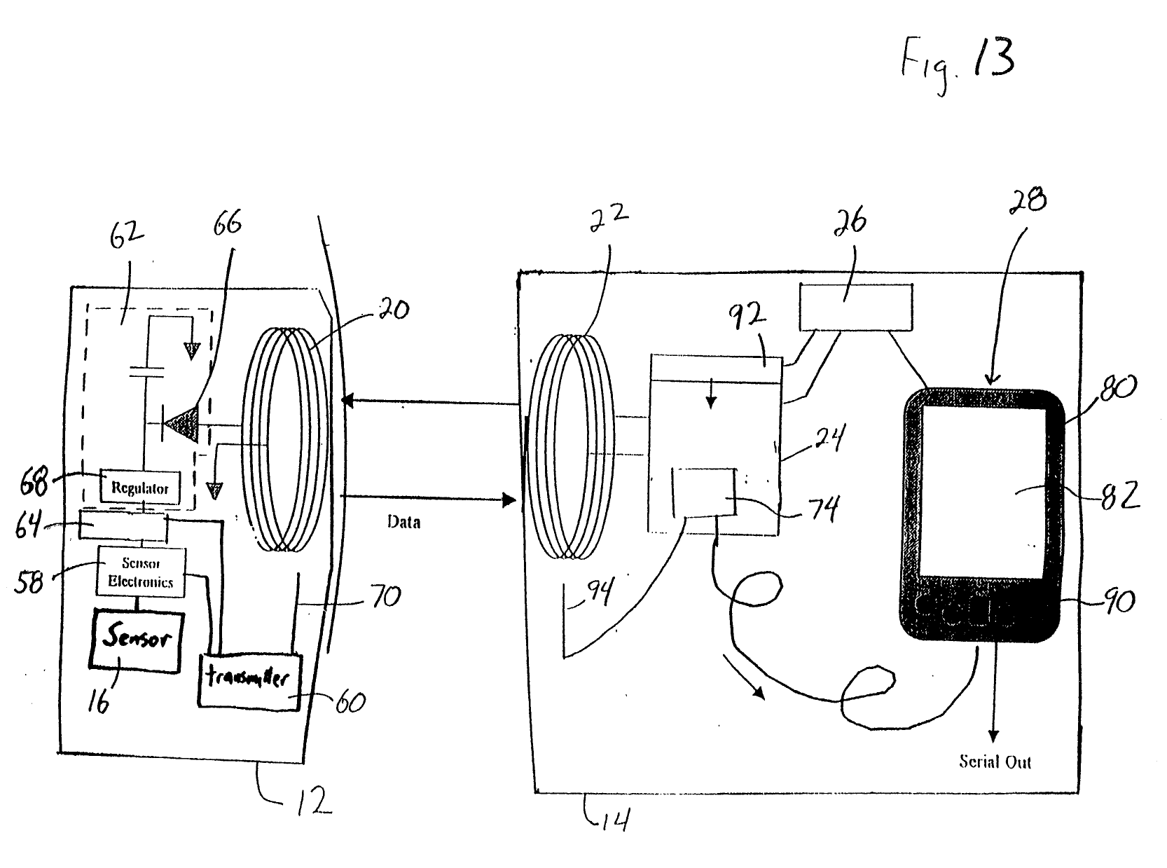

In this embodiment, probe coil 20 performs both the function of inductively coupling with external coil 22 to receive power from external device 14 and transmitting information from transmitter 60 to external device 14. In an alternate embodiment shown in FIG. 13, these two functions are separated. In this alternate embodiment, probe coil 20 performs only the function of inductively coupling with external coil 22 to receive power from external device. But, a probe antenna 70 is provided that serves the function of transmitting parameter information from the transmitter 60 to the external device 14.

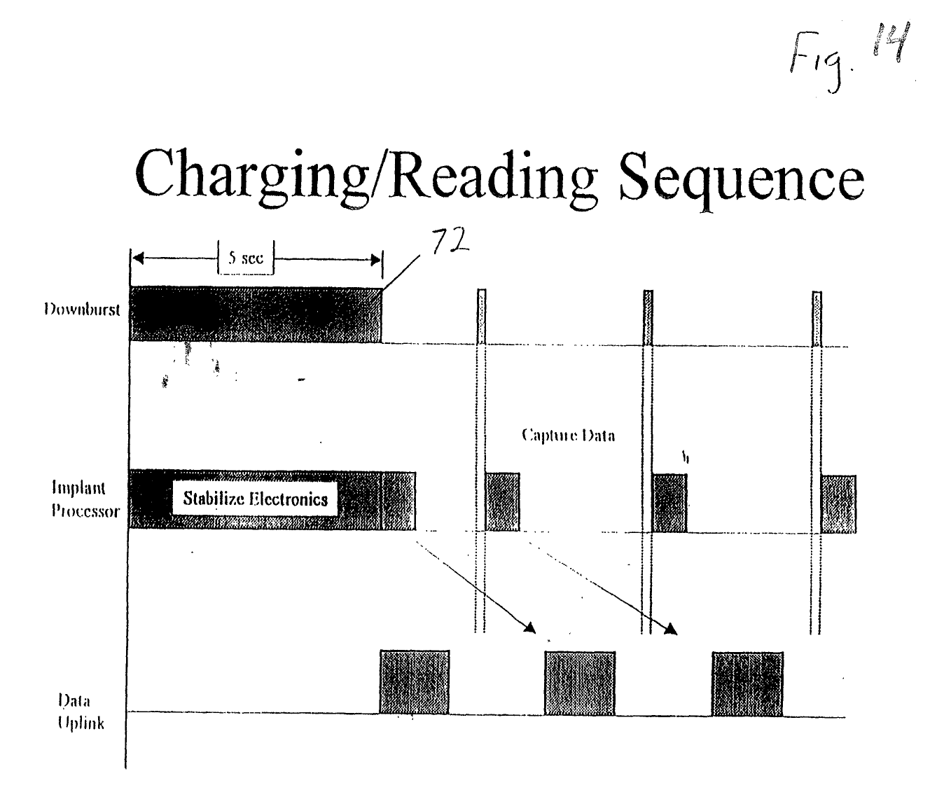

In the embodiment of FIG. 12, as shown in FIG. 14, when probe coil 20 is inductively coupled to external coil 22, probe coil 20 receives a downburst of energy 72 from the external device 14 through the external coil 22. The downburst of energy 72 preferably lasts for a specified time period to allow the temporary energy source 64 to be charged, for example, about 5 seconds, although more or less time may be used as desired. This downburst of energy 72 is converted to a regulated DC voltage by rectifier 66 and regulator 68 and charges the temporary energy source 64 to provide temporary energy to the probe electronics 18 as described above.

When the probe coil 20 is inductively coupled to the external coil 22 and the probe 12 is receiving power from the external device 14 or after the temporary energy source 64 is charged, sensor electronics 58 directs sensor 16 to sense the parameter and communicate the sensed parameter to the sensor electronics 58. Sensor electronics 58 processes the sensed parameter information and passes it to the transmitter 60 where the parameter information is converted into a form capable of being sent via telemetry from the transmitter 60 to the external device 14. The parameter information is then sent from the transmitter 60 and either the probe coil 20 acting as an antenna or the probe antenna 70, to the external device 14. External device 14 receives the transmitted parameter information through the external coil 22 acting as an antenna or the external device antenna 94, and a receiver 74, preferably located within external device 14.

This process of sensing parameters and transmitting it to the external device 14 may be continued for as long as the probe 12 receives power from the external device 14 or as long as the temporary energy source 64 has power or for a lesser time if desired. If sufficiently many occurrences of sensing parameters and communicating the sensed parameters are desired so that the power capacity of the temporary energy source 64 is or will be exceeded, power may again be downloaded from the external device 14 as described above. As a result, the temporary energy source 64 will be recharged and the sensing and transmitting process continued as described above.

The embodiment of probe 12 is a passive system without a long-term power source on the probe 12. As a result, probe 12 is a relatively low-cost device for measuring and communicating parameters. This embodiment allows a “real time” snapshot of the parameter of interest.

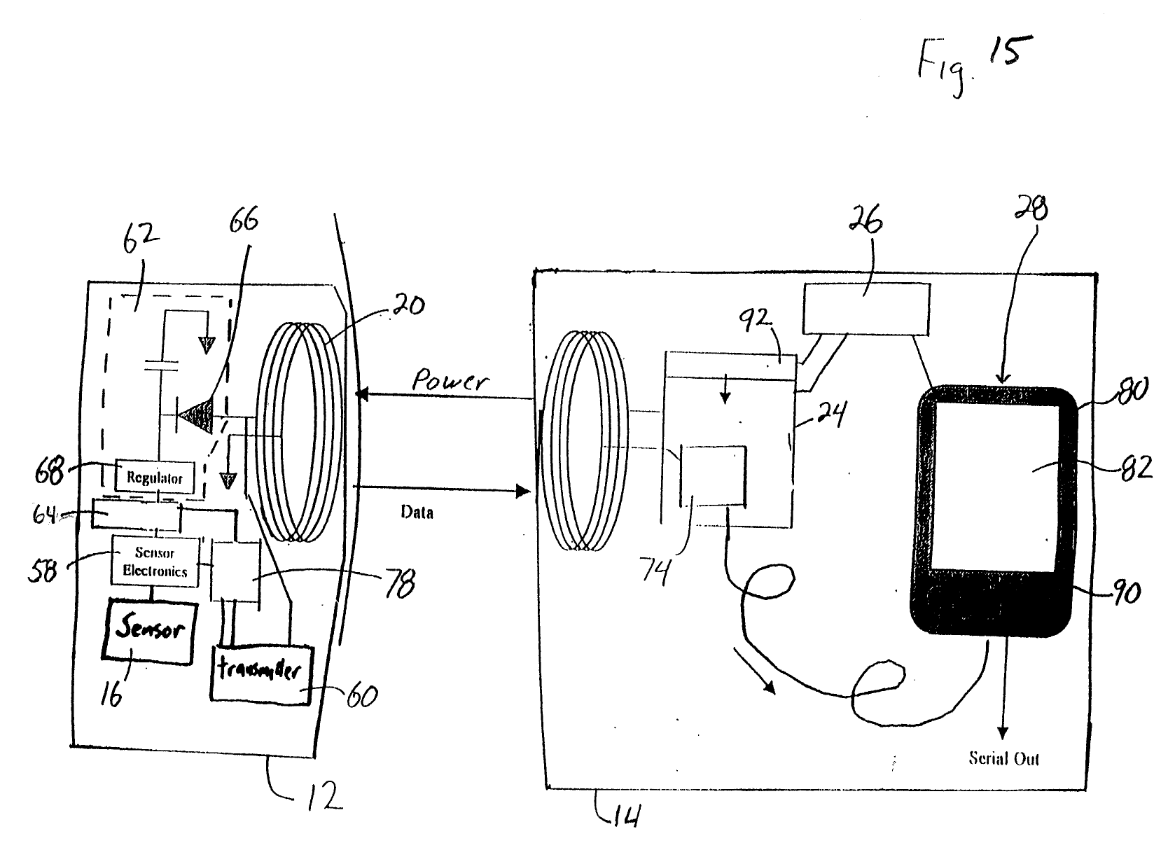

Alternately, as shown in FIG. 15, a long-term power source 76 may be provided to power the probe electronics 18. Long-term power source 76 may take the form of a battery that may or may not be rechargeable or a power capacitor such as a “super-capacitor” as is well understood in the art. Long-term power source 76 must have a capacity sufficient to power the probe 12 for a relatively long time. Where the long-term power source 76 is used, the temporary energy source 64 is replaced with the long-term power source 76. Where a storage device 78 is present, as will be explained hereafter, long-term power source 76 may also provide power to storage device 78 as well.

In this embodiment (FIG. 11), external device 14 includes an external coil 22, external electronics 24, a power source 26, and a user communication system 28. Power source 26 provides power to operate the external electronics 24 and user communication system 28 and provides power to external coil 22 that will be passed to probe 12 through inductive coupling with probe coil 20. Power source 26 may be either a battery or ordinary line current that has been adapted to provide power by such means as rectifying and filtering line AC power to produce a DC voltage as is well understood in the art.

External electronics 24 preferably contains a receiver 74 although the receiver 74 may be a separate component connected to the external device 14. Receiver 74 receives and processes the parameter information transmitted by transmitter 60 and received by external coil 22 acting as an antenna.

External device 14 may also include an ambient parameter sensor 92. Ambient parameter sensor 92 measures ambient parameters such as pressure, temperature and humidity. Where ambient parameter sensor 92 is a barometer, barometer 92 measures the atmospheric pressure. This measured atmospheric pressure is then subtracted from the pressure measured by sensor 16 and transmitted from probe 12 to external device 14 to produce the “gauge” pressure. This “gauge” pressure is independent of the ambient atmospheric pressure, which is influenced by weather systems and altitude. Of course, where ambient parameter sensor 92 measures temperature or humidity, ambient parameter sensor 92 will be a thermometer or a hygrometer as is well understood in the art. Further ambient parameters will occur to those skilled in the art and are intended to be included in the scope of the ambient parameter sensor 92. This ambient parameter information may be displayed directly, used to calibrate the sensor 16 or processed with parameter information from sensor 16.

In one embodiment, external coil 22 serves both to couple with the probe coil 20 to provide power to the probe 12 and as an antenna to receive information transmitted by probe 12 from transmitter 60. As such, external coil 22 is connected to receiver 74.

Alternately, an external device antenna 94 may be present, separate from external coil 22. In this embodiment, external device antenna 94 is connected to receiver 74 and communicates with probe antenna 70 or probe coil 20 to receive information transmitted from transmitter 60. In this embodiment, external coil 22 is not connected to receiver 74.

To use the device 10 to measure a parameter of the body, the first step is cut through the skin and place the sensor 16. For example, if the device 10 is to be mounted on a skull 50 and the sensor 16 placed inside a head, after the skin is opened and the skull exposed, a hole 52 is preferable drilled in the skull 50 to allow the sensor 16 to be placed in the head. The probe 12 is then implanted as described above. Thereafter, the patient's skin is closed so that the probe 12 is entirely contained within the patient's skin.

The external device 14 is brought near the probe 12 so that the probe coil 20 is inductively coupled to the external coil 22 and power is transferred from the external device 14 to the probe 12.

The hole 52 is then sealed with the probe 12 in place. Thereafter, the patient's skin is re-connected over the probe 12 where the wound will heal. This will seal the probe 12 underneath the patient's skin.

When a measurement of a parameter is desired, the external device 14 is placed with its external coil 22 over the probe coil 20. The probe 12 is powered by transmitting a downburst of energy 72 from the external device 14. In this embodiment, the initial downburst of energy 72 after power-up lasts about 5 seconds. This allows the probe electronics 18 to stabilize and set up such things as internal clocks, etc. Subsequent downbursts of energy 72 are preferably about 2 ms long.

In one embodiment, the probe 12 does not have an on-site battery. Therefore, on power-up the probe electronics 18 performs an autocalibration operation to ensure that the parameter measurements by sensor 16 will fall within the range of the probe electronics 18. Whenever the falling edge of the downburst of energy 72 is detected, the probe 12 uplinks its sensed parameter measurements to the external device 14. The uplink continues for as long as the external device 14 sends downbursts of energy 72 to the probe 12. The frequency of uplink is controlled by the external device 14 and cannot exceed the rate of downburst of energy 72. It is also possible to periodically uplink the stored calibration coefficients to the external device 14 or to uplink the stored calibration coefficients to the external device 14 with every uplink of sensed parameters.

In one embodiment, each uplink of the sensed parameter measurements is transmitted from probe 12 to external device 14 multiple times, for example thrice, to compensate for telemetry or processing errors. In a continuous mode, when the external device 14 intermittently sends downbursts of energy 72 to provide essentially continuous power to probe 12 and receives uplinked parameter measurements.

In addition, as explained above, calibration coefficients for sensor 16 may be stored in the probe 12 in the probe electronics 18, storage device 78 or microprocessor 102. Where these calibration coefficients are stored in probe 12, these coefficients may be uplinked from the probe 12 to the external device for the purpose of post-measurement processing to achieve to achieve an accurate report of the parameters measured by sensor 16. These coefficients may be uplinked to external device 14 when probe 12 is first powered up or may be uplinked with every uplink of sensed parameters.

Further, data such as the serial number or model number of the probe 12 may be stored in probe 12 in the probe electronics 18, storage device 78 or microprocessor 102 or in the external device 14. Where such serial number or model number is stored in probe 12, this information may be uplinked to external device 14 when probe 12 is first powered up or may be uplinked with every uplink of sensed parameters.

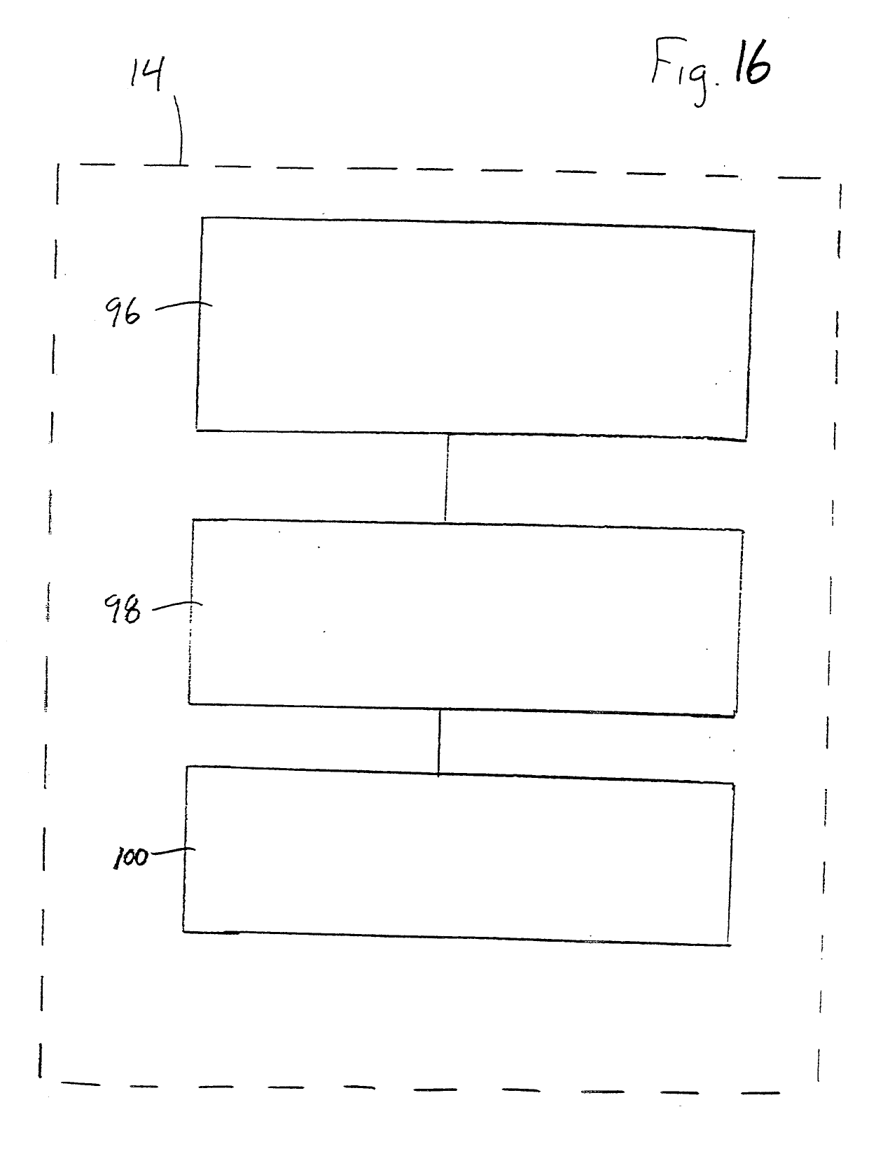

In one embodiment, external device 14 is a single unit that includes the components of an external coil 22, external electronics 24, a power source 26, user communication system 28 and an external device antenna 94, if present. However, external device 14 may be two or more separate devices. For example, as shown in FIG. 16, one device 96 may provide power to the probe 12 through inductive coupling between the probe coil 20 and external coil 22, a second device 98 may receive the parameter information transmitted by transmitter 60 and a third device 100 may display the parameter information received by the second device 98.

In one embodiment where probe 12 includes a passive system 24, it is critical that probe coil 20 and external coil 22 be coupled to allow power to be passed from the external device 14 to the probe 12 and for parameter information to be passed from probe 12 to external device 14. It may be desirable to have an audible confirmation that probe coil 20 and external coil 22 are coupled. This may be accomplished by probe 12 uploading a signal to external device 14 indicating that probe coil 20 and external coil 22 are inductively coupled. This signal may be used by the external device 14 to trigger an audible signal indicating that the probe 12 and the external device 14 are inductively coupled.

Alternately, the loading of the external coil 22 caused by the inductive coupling with the probe coil 20 can be detected by the external device 14 and used to determine coupling efficiency. This loading can be detected by monitoring the power passed through the external coil 22. As the inductive coupling between the external coil and probe coil 20 increases, the power passing through the external coil 22 to the probe coil 20 will increase. By monitoring this power and comparing instantaneous power measures, trends in power transmission (i.e., increasing or decreasing) or a relative maximum power transmission amount may be determined. This information can be used to determine whether increasingly efficient coupling positions between external coil 22 and probe coil 20 are being reached or that the optimum coupling position has been reached.

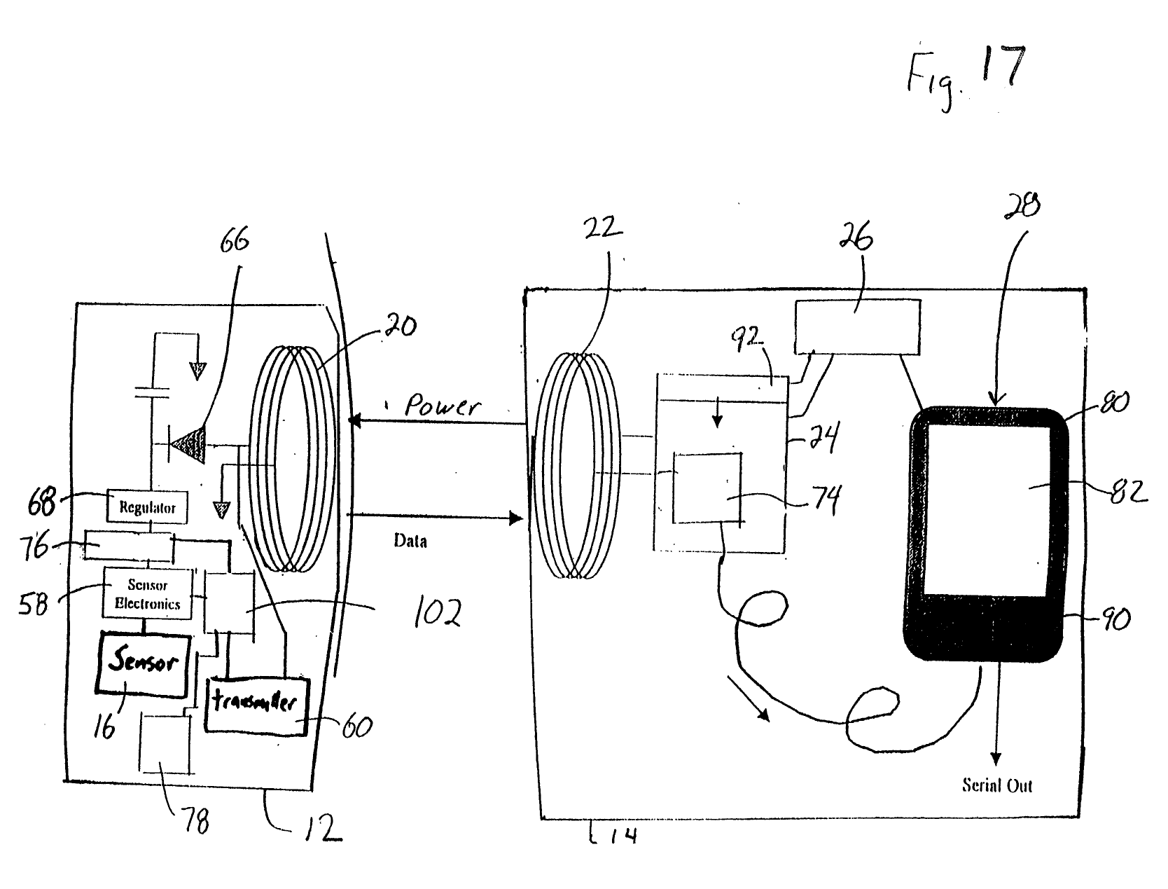

Further, it may be desirable to store sensed parameter information from the sensor 16 to be transmitted from transmitter 60 at a later time. This may be accomplished by having a storage device 78 attached to sensor 16 and transmitter 60 (FIG. 17). Storage device 78 may be of the type disclosed in U.S. Pat. No. 5,817,137 entitled “Compressed Patient Narrative Storage In and Full Text Reconstruction from Implantable Medical Devices”, issued on Oct. 6, 1998 to William F. Kaemmerer or U.S. Pat. No. 5,549,654 entitled “Interactive Interpretation of Event Markers in Body-Implantable Medical Devices” issued to Richard M. Powell on Aug. 27, 1996, both assigned to the assignee of the present application, the teachings of which are incorporated herein by reference in their entirety.

Storage device 78 may be located in probe head 36, electronics case 41 or body 48 or may be located separate from but electrically connected to the probe 12. For example, storage device 78 may be located near the clavicle in a manner similar to the placement of the Reveal® cardiac recording device manufactured and sold by Medtronic, Inc. of Minneapolis, Minn. Further, storage device 78 may be directly connected to probe 12 by wires, through the “body bus” communication system described above or other similar communication means.

In this embodiment, probe 12 requires a long-term power source 76 to provide power to the sensor 16, sensor electronics 58 and the storage device 78. Sensor electronics 58 would periodically direct sensor 16 to sense the parameter of interest. Alternately, sensor electronics 58 could be directed from a signal from the external device 14 to direct sensor 16 to sense parameter information.

In either case, the sensed parameter would then be communicated to the storage device where it would be stored. Then, either periodically or when an inquiry is made from the external device 14, parameter information would be uploaded from the storage device 78 through the transmitter 60 to the external device 14 as described above.

In an alternate embodiment (FIG. 18), sensor electronics 28 and storage device 78 may be connected to a microprocessor 102 as shown in FIG. 13. In this embodiment, parameter measurements may be processed by microprocessor 102 before being stored in storage device 78. Alternately, microprocessor 102 may take a series of stored measurements from storage device 78 and process the series, as for example, to produce a running average of the parameter values. Such processed information may by transmitted from probe 12 to external device 14 at the time of the processing or may be stored in storage device 78 to be transmitted to external device 14 at a later time.

The sensed parameter information may also be used to control or activate a control device 104. In this embodiment, shown schematically in FIG. 18, the control device 104 may control a pump or valve in a CSF drainage system, a drug delivery system or an electrical stimulation device, to name but a few examples. In the embodiment shown, the control device 104 is connected to microprocessor 102 so that microprocessor 102 controls whether the control device 104. In use, where microprocessor 102 has determined that a parameter sensed by sensor 16 is within a predetermined range, microprocessor 102 activates the control device 104 to control the corresponding medical device. When the microprocessor 102 has determined that the sensed parameter from sensor 16 is outside the predetermined range, microprocessor 102 causes control device 104 to either cease or diminish operation of the control device 104.

For example, as shown in the embodiment of FIG. 18, a drainage catheter 2 is placed in the ventricle 4 of a patient, coupled to the control device 104. The control device 104 is preferably connected to a peritoneal or atrial catheter 8 although the control device 104 could be connected to a drainage bag. Control device 104 may be a pump or a valve connected between the drainage catheter 2 and the peritoneal or atrial catheter 8 or drainage bag. Where the control device 104 is a pump, the pump pumps CSF fluid from the ventricle 4 to the peritoneal or atrial catheter 8 where it is absorbed into the body or into a drainage bag. Where the control device 104 is a valve, the valve, when open, allows CSF fluid to drain from the ventricle 4 through the peritoneal or atrial catheter 8 or into the drainage bag.

In the embodiment shown, the control device 104 is connected to microprocessor 102 so that microprocessor 102 controls whether the pump pumps CSF fluid or the valve is open to allow the drainage of CSF fluid.

In use, where microprocessor 102 has determined that the parameter sensed by sensor 16 is outside the predetermined range, microprocessor 102 activates the control device 104 to either pump CSF fluid or to open the valve to allow the excess CSF fluid to drain from the patient's ventricle. When the microprocessor 102 has determined that the sensed parameter is within a predetermined range, microprocessor 102 causes control device 104 to either cease pumping CSF fluid or closes the valve so that CSF ceases to drain through the valve.

The control device 104 may also control the operation of, for example, an adjustable subcutaneously implantable fluid flow valve in a CSF shunt system. Such a device is the Strata® Valve Adjustable Valve manufactured and sold by Medtronic—PS Medical of Goleta, Calif. and as disclosed in U.S. Pat. No. 5,637,083 entitled “Implantable Adjustable Fluid Flow Control Valve”, issued on Jun. 10, 1997 to William J. Bertrand and David A. Watson. Such an adjustable valve is useful in a physiological shunt system for controlling the flow of fluid from one part of the body to another such as from the patient's ventricle to the patient's ventricle or atrium. The control device 104, for example, controls the movement of an external or percutaneously-applied magnetic field, to cause the valve to provide a variety of pressure or flow characteristics.

In a variant of the embodiment using the control device 104 to control a valve or pump, the control device may also control another medical device such as a pacemaker, neurological electrical stimulator or a drug pump.

In addition, in one embodiment, the probe electronics 18 are located in the electronics case 42. In an alternate embodiment, the probe electronics 18 may be located in the body 48 or in the probe head 36.

One advantage of the device 10 described herein is that long-term monitoring of a parameter can be conveniently performed without risk of infection since the organ or tissue of interest, for example, the brain, is exposed only once during implantation. Thereafter, the probe 12 is encased within the skin of the patient where it can measure and communicate the parameter of interest.

An advantage of one embodiment of the present invention, is that the device does not have an on-site battery. Therefore, the probe 12 must run a start-up sequence each time power is transferred to the probe 12. In the present invention, this start-up sequence involves running an auto-calibration algorithm. This auto-calibration algorithm ensures that the pressure measurements received from the sensor 16 will always be within the desired range of the probe electronics 18.

It is also clear from the description above that the invention includes a method for measuring and communicating a parameter of a brain, tissue or other organs of a body. Without further limiting such methods described above, the method, in one embodiment, comprising the steps of:

providing:

an implantable first sensor 16 capable of measuring and communicating parameters of a brain, tissue or other organs; and

an activation system 15 connected to and responding to sensor 16, the activation system 15 having an external device capable of transmitting power to the sensor and receiving parameter information from the sensor;

cutting through the skin;

placing the sensor 16 at a desired location within the body;

closing the skin is closed so that the sensor 16 is entirely contained within the patient's skin;

bringing the external device 14 near the sensor 16 so that power is transferred from the external device 14 to the sensor 16; and

uplinking information from the sensor 16 to the external device 14.

The description contained herein is intended to be illustrative of the invention and not an exhaustive description. Many variations, combinations and alternatives to the disclosed embodiments will occur to one of ordinary skill in this art. Further, where specific values have been given, these values are intended to be illustrative of the invention and are not intended to be limiting. Further, specific anatomical locations for implanting the device have been described. These are intended to be exemplary and not limiting. The locations where the described device can be located will readily occur to those skilled in the art. All these alternatives, combinations and variations are intended to be included within the scope of the attached claims. Those familiar with the art may recognize other equivalents to the specific embodiments described herein which equivalents are also intended to be encompassed by the claims attached hereto.

1. A device for measuring and communicating parameters of a brain, tissue or other organs of a body comprising:
an implantable first sensor 16 capable of measuring and communicating parameters of a brain, tissue or other organs; and
an activation system 15 connected to and responding to sensor 16.

2. The device of claim 1 wherein the activation system 15 includes a user communication system 28 that communicates information from or about sensor 16 or may communicate alarms or other information based on the values that sensor 16 detects.

3. The device of claim 2 wherein the activation system 15 includes an external device 14, the external device 14 including an external coil 22, external electronics 24 and a power source 26.

4. The device of claim 2 wherein the external device 14 includes an ambient parameter sensor 92.

5. The device of claim 2 wherein the user communication system 28 includes a transmitter 60 and a receiver 74.

6. The device of claim 5 wherein the activation system 15 includes an external device 14 and wherein the receiver 74 is located in the external device 14.

7. The device of claim 5 wherein the user communication system 28 includes a display system 80 that displays or otherwise communicates the parameter information from the sensor 16 transmitted by the transmitter 60 to the receiver 74 to a user.

8. The device of claim 2 wherein the user communication system 28 includes a display system 80 that displays the parameter information sensed by the sensor 16 to the physician or other user.

9. The device of claim 2 wherein the user communication system 28 includes an alarm 90 that is triggered to alert the user to a parameter sensed by the sensor 16 that is outside of a pre-determined range.

10. The device of claim 1 wherein the activation system 15 and sensor 16 are co-located in the body of a patient.

11. The device of claim 1 wherein the sensor 16 and at least a portion of the activation system 15 are both implanted in a patient but are not co-located.

12. The device of claim 1 further comprising a control device 104 capable of taking action to effect the body of a patient wherein the activation system 15 directs the control device 104 to take action based on the information from or about sensor 16 or processed from information provided by sensor 16.

13. The device of claim 1 wherein the sensor 16 is chosen from the group consisting of piezoelectric activity sensors, intracranial pressure sensors, ecg sensors, ultrasound sensors, acoustic sensors, infrared sensors, air flow sensors, strap-type respiration sensors, CO₂ sensors, thermistor sensors, microwave Doppler sensors, inductance change respiratory electrodes, temperature sensors, eeg sensors, pH sensors, emg sensors, O₂, respiration sensors, electrolyte concentration, glucose sensors, blood pressures sensors, heart rate sensors, posture sensors and activity sensors.

14. The device of claim 1 further comprising a probe 12.

15. The device of claim 14 wherein the probe 12 includes sensor 16, probe electronics 18 and a probe coil 20.

16. The device of claim 15 further comprising an external device 14 and wherein the probe electronics 18 transfers power from the external device 14 to probe 12 to power probe 12 and uplinks sensed parameters from probe 12 to external device 14.

17. The device of claim 15 therein the probe electronics 18 includes an AC/DC conversion system 62.

18. The device of claim 17 wherein the probe coil 20 is connected to the AC/DC conversion system 62.

19. The device of claim 17 wherein the AC/DC conversion system 62 includes a rectifier 66 and a regulator 68 wherein the rectifier 66 is connected to probe coil 20 and converts AC power received from the probe coil 20 to DC power.

20. The device of claim 15 wherein the probe electronics 18 further includes a temporary energy source 64 that provides the energy to power the probe electronics 18.

21. The device of claim 20 wherein the temporary energy source 64 is chosen from the group consisting of a rechargeable battery, a non-rechargeable battery or a power capacitor such as a “super capacitor”.

22. The device of claim 15 wherein probe electronics 18 further includes a long-term power source 76 that provides the energy to power the probe electronics 18.

23. The device of claim 22 wherein the long-term power source 76 is chosen from the group consisting of a rechargeable battery, a non-rechargeable battery or a power capacitor such as a “super-capacitor”.

24. The device of claim 15 wherein the probe 12 includes an electronics case 42 and wherein the probe electronics 18 is stored in the electronics case 42.

25. The device of claim 24 wherein the electronics case 42 has a periphery 44, wherein screw threads are placed on the periphery 44 of the electronics case.

26. The device of claim 15 wherein the probe electronics 18 includes sensor electronics 58 and a transmitter 60 wherein the sensor electronics 58 is connected to sensor 16 and provides power to sensor 16, directs sensor 16 to take measurements and processes the sensed measurement signal from sensor 16.

27. The device of claim 26 wherein the transmitter 60 is connected to sensor electronics 58 and a probe coil 20 wherein the probe coil 20 acts as an antenna and wherein the transmitter 60 and probe coil 20 communicate parameter information determined by sensor 16 to an external device 14 by telemetry.

28. The device of claim 26 further comprising a microprocessor 102 and a storage device 78 and wherein the sensor electronics 28 and storage device 78 are connected to the microprocessor 102 whereby parameter measurements by the sensor 16 are processed by microprocessor 102 before being stored in storage device 78.

29. The device of claim 28 further comprising a control device 104 connected to microprocessor 102 and wherein the sensed parameter information controls or activates the control device 104.

30. The device of claim 29 wherein the control device 104 is chosen from the group consisting of a pump, a valve in a CSF drainage system, a drug delivery system or an electrical stimulation device.

31. The device of claim 15 wherein the probe electronics 18 further includes a temporary energy source 64 and wherein the probe coil 20 is connected to an AC/DC conversion system 62 and wherein the probe coil 20 is inductively coupled to an external coil 22 in the external device 24 and wherein the temporary energy source 64 is connected to AC/DC conversion system 62 whereby inductive coupling between probe coil 20 and external coil 22 provides power to the probe coil 20 and through AC/DC conversion system 62, charges up the temporary energy source 64 and whereby the temporary energy source 64 then provides the energy to power the probe electronics 18.

32. The device of claim 14 wherein the probe 12 has a proximal end 30 and a distal end 32 and a central axis 34 and wherein the sensor 16 is located at the distal end 32.

33. The device of claim 32 wherein the probe 12 includes a probe head 36 located at the proximal end 30, the probe head 36 having an outer edge 40.

34. The device of claim 33 wherein the probe head 36 is roughly discoid in shape.

35. The device of claim 34 wherein the probe head 36 includes an embedded probe coil 20 and wherein the probe coil 20 is an inductive coil.

36. The device of claim 35 wherein the probe coil 20 is wound around axis 34 in the plane of probe head 36.

37. The device of claim 33 wherein screw threads are placed on the outer edge 40 of probe head 36.

38. The device of claim 33 further comprising a storage device 78 located in probe head 36.

39. The device of claim 14 wherein the sensor 16 is separated from the probe 12.

40. The device of claim 39 further comprising a body 48 having a proximal and a distal end, the proximal end of the body 48 connected to the probe 12, wherein the sensor 16 is located at the distal end of the body 48.

41. The device of claim 40 wherein the body 48 is rigid.

42. The device of claim 40 wherein the body 48 is flexible.

43. The device of claim 39 wherein the sensor 16 is connected to the probe 12 through a body bus.

44. The device of claim 14 wherein the probe 12 has a proximal end 30, a distal end 32 and a central axis 34 wherein the probe 12 includes a probe head 36 located at the proximal end 30.

45. The device of claim 44 further comprising a body 48 having a proximal and a distal end, the proximal end of the body 48 connected to the probe head 36, wherein the sensor 16 is located at the distal end of the body 48.

46. The device of claim 45 wherein the body 48 is rigid.

47. The device of claim 45 wherein the body 48 is flexible.

48. The device of claim 1 wherein the sensor 16 is connected to the activation system 15 through a body bus.

49. The device of claim 1 further comprising a second sensor.

50. The device of claim 1 further comprising a microprocessor 102 and a storage device 78 wherein the sensor 16 and storage device 78 are connected to the microprocessor 102 whereby parameter measurements by the sensor 16 are processed by microprocessor 102 before being stored in storage device 78.

51. The device of claim 50 further comprising a control device 104 connected to microprocessor 102 and wherein the sensed parameter information controls or activates the control device 104.

52. The device of claim 51 wherein the control device is chosen from the group consisting of a pump, a valve in a CSF drainage system, a drug delivery system or an electrical stimulation device.

53. A device for measuring and communicating parameters of a brain, tissue or other organs of a body comprising:
an implantable first sensor 16 capable of measuring and communicating parameters of a brain, tissue or other organs;
an activation system 15 connected to and responding to sensor 16, the activation system 15 including a user communication system 28 that communicates information from or about sensor 16 or may communicate alarms or other information based on the values that sensor 16 detects;
a probe 12 having a proximal end and a distal end, wherein the probe 12 includes sensor 16, probe electronics 18 and a probe coil 20, wherein the probe electronics 18 includes sensor electronics 58 and a transmitter 60 wherein the sensor electronics 58 is connected to sensor 16 and provides power to sensor 16, directs sensor 16 to take measurements and processes the sensed measurement signal from sensor 16, wherein the probe 12 includes a probe head 36 located at the proximal end 30, the probe head 36 having an outer edge 40, wherein the sensor 16 is located at the distal end of the probe 12.

54. A device for measuring and communicating parameters of a brain, tissue or other organs of a body comprising:
an implantable first sensor 16 capable of measuring and communicating parameters of a brain, tissue or other organs;
an activation system 15 connected to and responding to sensor 16, the activation system 15 including a user communication system 28 that communicates information from or about sensor 16 or may communicate alarms or other information based on the values that sensor 16 detects;
a probe 12 having a proximal end and a distal end, wherein the probe 12 includes sensor 16, probe electronics 18 and a probe coil 20, wherein the probe electronics 18 includes sensor electronics 58 and a transmitter 60 wherein the sensor electronics 58 is connected to sensor 16 and provides power to sensor 16, directs sensor 16 to take measurements and processes the sensed measurement signal from sensor 16, wherein the probe 12 includes a probe head 36 located at the proximal end 30, the probe head 36 having an outer edge 40, wherein the sensor 16 is located at the distal end of the probe 12, wherein the probe head 36 is roughly discoid in shape and includes an embedded probe coil 20 and wherein the probe coil 20 is an inductive coil, wherein the probe coil 20 is wound around axis 34 in the plane of probe head 36; and,
a storage device 78 located in probe head 36.

55. A device for measuring and communicating parameters of a brain, tissue or other organs of a body comprising:
an implantable first sensor 16 capable of measuring and communicating parameters of a brain, tissue or other organs;
an activation system 15 connected to and responding to sensor 16, the activation system 15 including a user communication system 28 that communicates information from or about sensor 16 or may communicate alarms or other information based on the values that sensor 16 detects;
a probe 12 having a proximal end and a distal end, wherein the probe 12 includes sensor 16, probe electronics 18 and a probe coil 20, wherein the probe electronics 18 includes sensor electronics 58 and a transmitter 60 wherein the sensor electronics 58 is connected to sensor 16 and provides power to sensor 16, directs sensor 16 to take measurements and processes the sensed measurement signal from sensor 16, wherein the probe 12 includes a probe head 36 located at the proximal end 30, the probe head 36 having an outer edge 40, wherein the probe head 36 is roughly discoid in shape and includes an embedded probe coil 20 and wherein the probe coil 20 is an inductive coil, wherein the probe coil 20 is wound around axis 34 in the plane of probe head 36;
a storage device 78 located in probe head 36; and.
a body 48 having a proximal and a distal end, the proximal end of the body 48 connected to the probe 12, wherein the sensor 16 is located at the distal end of the body 48.

56. The device of claim 55 wherein the body 48 is rigid.

57. The device of claim 55 wherein the body 48 is flexible.

58. A method for measuring and communicating a parameter of a brain, tissue or other organs of a body comprising the steps of:
providing a device for measuring and communicating parameters of a brain, tissue or other organs of a body comprising:
an implantable first sensor 16 capable of measuring and communicating parameters of a brain, tissue or other organs; and
an activation system 15 connected to and responding to sensor 16, the activation system 15 having an external device capable of transmitting power to the sensor and receiving parameter information from the sensor;
cutting through the skin;
placing the sensor 16 at a desired location within the body;
closing the skin is closed so that the sensor 16 is entirely contained within the patient's skin;
bringing the external device 14 near the sensor 16 so that power is transferred from the external device 14 to the sensor 16;
uplinking information from the sensor 16 to the external device 14.
